(19) United States
(12) Patent Application Publication
(10) Pub. No.: US 2024/0316360 A1
(43) Pub. Date: Sep. 26, 2024

(54) LOCKING INTRA-ORAL DEVICE FOR PROTECTING ORAL TISSUES DURING RADIATION TREATMENT

(71) Applicant: POLL Medical LLC, Beaverton, OR (US)

(72) Inventors: Adrian Abram Polliack, Lake Oswego, OR (US); Robert Henry Niemeyer III, Tigard, OR (US); Christopher Albert Jacobs, West Linn, OR (US)

(21) Appl. No.: 18/581,368

(22) Filed: Feb. 19, 2024

Related U.S. Application Data
(60) Provisional application No. 63/453,974, filed on Mar. 22, 2023.

(51) Int. Cl. A61N 5/10

(52) U.S. Cl. CPC A61N 5/10; A61N 2005/1094

(57) ABSTRACT

An intra-oral device for positioning oral tissues in a patient includes an upper dental arch tray and lower dental arch tray configured for engagement with the teeth or edentulous arches of the patient. A compression fitting is disposed between the upper and lower dental arch trays and a ball disposed and forming a rotatable ball joint within the compression fitting. An elongate post of a protective element is received through an aperture formed through the ball protective element, and a head of the protective element is configured to be extensible into the mouth of the patient and rotatable via the ball joint through a plurality of positions against a tongue of the patient. The position of the protective element is lockable in place via the compression fitting about the ball.

### CROSS-REFERENCES TO RELATED APPLICATIONS

This application claims the benefit of U.S. Provisional Patent Application No. 63/453,974, filed Mar. 22, 2023, whose contents are incorporated herein for all purposes.

### BACKGROUND OF THE INVENTION

### Field of the Invention

This application relates to devices for reducing exposure to and immobilization of certain tissues to ionizing radiation during treatment of head and neck cancers in patients, and to improved methods for use of such devices.

### Description of the Prior Art

As is generally known, cancer treatment can be complex and invasive. In some cases, patients need to be subjected to high-dose radiation treatment to eradicate and prevent additional cancerous tissue growth. In cases of head and neck cancer, cancerous tissue growth is typically located on the floor of the mouth, cheek lining, tonsils, pharynx, the tongue, and/or lymph nodes but could also include other areas. When radiation treatment is applied in cases of head and neck cancer, some damage often occurs to non-cancerous tissues that lie in the pathway of the beam treating the cancer. For example, a patient's tongue may not be cancerous when the cancer is in the tonsils, but the tongue may be damaged by ionizing radiation that passes through the tongue to treat the tonsillar tumor. Thus, it would be advantageous if non-cancerous tissue could be spared radiation damage by altering the beam path, or when not possible, by moving such non-cancerous tissue physically out of the way of the radiation beam.

One such device used to mitigate exposure of a patient's tongue to ionizing radiation is described in U.S. Pat. No. 9,504,537. This intra-oral device comprises upper and lower dental arch members that a patient bites down upon and includes a paddle that extends through a front section of the arch members and into the interior of the mouth adjacent the tongue. The paddle may be moved laterally to push the tongue far to the right or left and out of the way of the beam of ionizing radiation. Although such a device has proven effective, it includes certain features that invite improvement.

One such feature in need of improvement is that the paddle is coupled to the dental arch via a stabilizing strut that is only slidingly received within a gimbal sandwiched between the dental arches. This type of coupling is cumbersome to assemble by the radiation oncology team during patient set up. This set up also means more device material is situated inside the patient's mouth. Finally, although small in likelihood, it could slip during use so that the tongue slides back into the path of the radiation beam. A more effective stabilizing method is required that better maintains its position within the patient's mouth while improving the adjustability and precision of the paddle in relation to the dental arches and minimizes device material that resides in the patient's mouth.

Accordingly, the need remains for design improvements to oral protective devices that includes better and potentially simpler means for locking the tongue manipulation device in place during use.

### SUMMARY OF THE INVENTION

An intra-oral device for positioning oral tissues in a patient comprises an upper dental arch tray configured for engagement with the maxillary teeth or maxillary edentulous arch of a patient, and a lower dental arch tray configured for engagement with the mandibular teeth or mandibular edentulous arch of the patient. A compression fitting is disposed between the upper and lower dental arch trays, and includes a ball disposed and forming a rotatable ball joint within the compression fitting, wherein ball has an aperture formed therethrough. A protective element is received through the aperture in the ball wherein a rod of the protective element is received through the aperture of the ball and a head is configured to be extensible into the mouth of the patient. The protective element is rotatable via the ball joint through a plurality of positions against a tongue of the patient and lockable in place via the compression fitting about the ball.

Aspects of the invention include various embodiments including a taper nut used in the compression fitting, whereby opposed exterior threaded portions are defined on each of the upper and lower arch trays. Hemispherical depressions are formed interior of the exterior threaded portions such that assembly of the upper and lower arch trays result in a substantially spherical depression configured to capture the ball within. The taper nut is configured to engage with the exterior threaded portions of the upper and lower arch trays and compress the trays together to thereby compress the substantially spherical depression against the ball and lock the rotation movement of the ball in place and rod within the ball aperture.

An alternate aspect of the invention includes a tension ring, whereby opposed interior threaded portions are defined on each of the upper and lower arch trays and first compression surfaces are located proximal the dental arches relative to the interior threaded portions. An exterior plate having exterior threaded surfaces thereon is configured to threadedly engage with the interior threaded portions of the upper and lower arch trays. This aspect further includes second compression surfaces proximal the first compression surfaces relative to the exterior threaded surfaces, wherein the first and second pressions surfaces form a substantially spherical depression therebetween. The tension ring is configured to engage around an exterior of the opposed interior threaded portions and prevent them from expanding as the exterior threaded surfaces are threaded into the interior threaded portions to contract the substantially spherical depression thereby longitudinally about the received ball and thereby lock the rotation movement of the ball in place and rod within the ball aperture.

Also described is a method for repositioning the tongue within the oral cavity of a patient with or without maxillary and/or mandibular teeth. The method comprises installing an intra-oral device in the mouth of the patient so that oral arches of the patient engage with an upper dental arch member and a lower dental arch member of the intra-oral device. A protective element is then moved within a ball joint coupled to a distal portion of the intra-oral device so that an extensible portion of the protective element bears proximally against the tongue of the patient and moves the tongue from a central axis of the oral cavity. The step of moving includes capturing a ball of the ball joint within a compressible fitting between the oral arches, wherein the ball includes an aperture therethrough passing from a proximal to a distal portion and extending a rod of the protective device through the aperture of the ball and rotating the protective element through the ball joint until the extensible portion is in a desired position adjacent a tongue of the patient. The fitting is then compressed about the ball so that a dimension of the ball is pinched within the compression fitting to thereby prevent the ball from rotating within the ball joint, and further pinching the aperture closed about the rod extended through the aperture to thereby fix the extensible portion in position within the mouth of the patient so as to secure the patient's tongue at a desired location while providing radiation treatment to selected tissues.

Additional features and advantages of embodiments of the present disclosure will be set forth in the description which follows, and in part will be apparent from the description, or may be learned by practice of embodiments of the present disclosure. The objectives and other advantages of the embodiments of the present disclosure may be realized and attained by the structure particularly pointed out in the written description and claims hereof as well as the appended drawings.

It is to be understood that both the foregoing general description and the following detailed description are exemplary and explanatory and are intended to provide further explanation of embodiments of the invention as claimed.

### BRIEF DESCRIPTION OF THE DRAWINGS

The foregoing and other objects, features and advantages of the invention will become more readily apparent from the following detailed description of a preferred embodiment of the invention that proceeds with reference to the accompanying drawings, in which:

FIG. 1 is a perspective view of an intra-oral device constructed according to a first embodiment of the invention.

FIG. 2 is a perspective view of an intra-oral device constructed according to a second embodiment of the invention.

FIG. 3 is an exploded perspective view of the intra-oral device shown in FIG. 1.

FIG. 4 is an exploded perspective view of the intra-oral device shown in FIG. 2.

FIG. 5 is a perspective view of the ball and reinforced post feature of the invention with the post removed from the ball.

FIG. 6 is a perspective view of the ball and reinforced post feature of the invention with the post received within the ball of FIG. 5.

FIG. 7 is a side elevation section view taken along line 7-7 of FIG. 1.

FIG. 8 is a front elevation section view taken along line 8-8 of FIG. 1 with the device tightened in a first position.

FIG. 9 is a front elevation section view taken along line 8-8 of FIG. 1 with the device tightened in a second (tighter) position.

FIG. 10 is a side elevation section view taken along line 10-10 of FIG. 2.

FIG. 11 is a front elevation section view taken along line 11-11 of FIG. 2 with the device tightened in a first position.

FIG. 12 is a front elevation section view taken along line 11-11 of FIG. 2 with the device tightened in a second (tighter) position.

FIG. 13 is top plan view of the device of FIG. 1 inserted within the mouth of a patient and operating to move the patient tongue to the right side of the mouth.

FIG. 14 is a side elevation view of FIG. 13 showing insertion of the device of FIG. 1 within the mouth of a patient.

### DETAILED DESCRIPTION

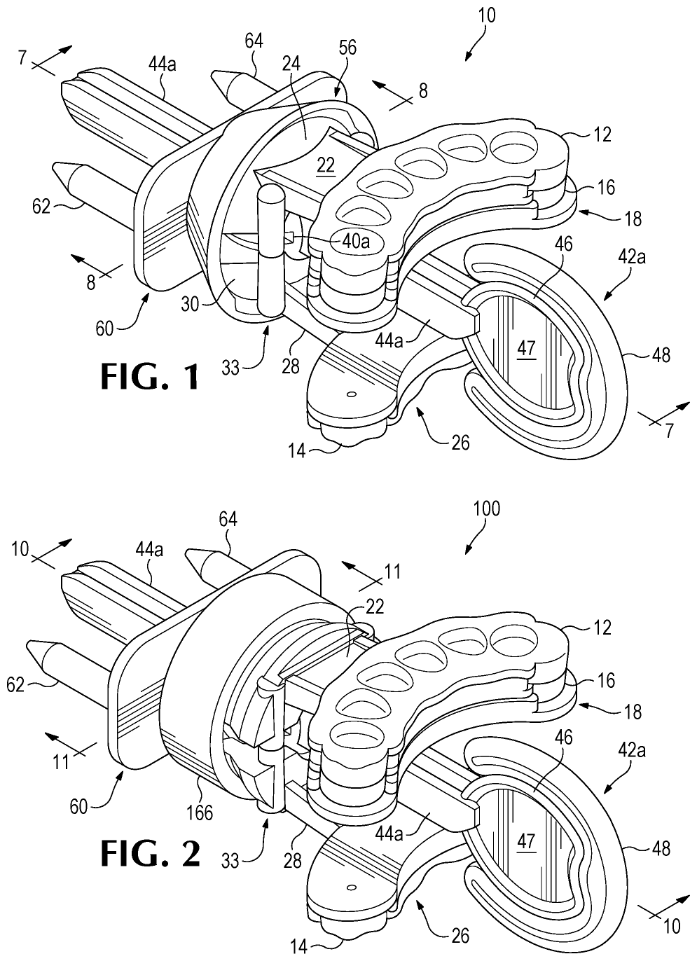

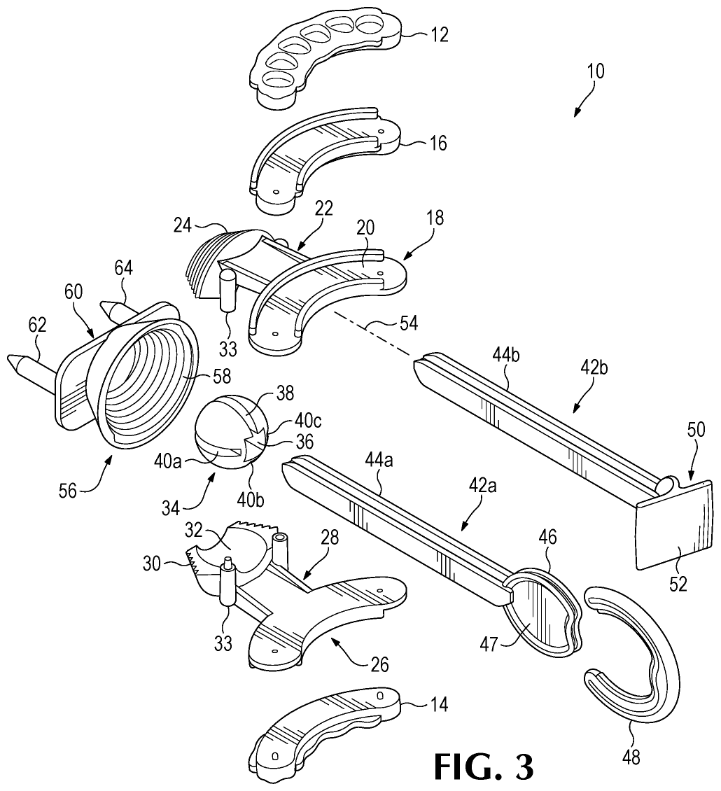

FIGS. 1 and 3 illustrate a first embodiment of an intra-oral device 10 configured according to the present invention with a taper nut forming a compressible fitting (described further below). Intra-oral device 10 includes an upper arch tray 12 upon which a patient bites down with at least a portion of their maxillary teeth or maxillary edentulous arch as shown in FIG. 14. Device 10 further includes an opposed lower arch tray 14 configured to receive at least a portion of the patient's mandibular teeth or mandibular edentulous arch, again as shown in FIG. 14. Upper arch tray 12 (as well as lower arch tray 14) is preferably formed of a solid block of elastomeric impression material such as EVA that sits within the upper and lower arch trays to form fit around and engage the biting surfaces of the teeth of the patient for a more comfortable and secure fit of the device 10 within the patient's mouth. If these trays 12, 14 are used as molded inserts into the device 10, they can be softened up by temporarily immersing in hot water and then reinserting into the receiving portions of the device so that the patient can then comfortably bite down on these softened elements.

An upper spacer 16 is optionally fitted between the upper arch tray 12 and upper tray mount 18 to increase the bite spacing between the upper and lower teeth. A lower spacer (not shown) is optionally fitted between the lower arch tray 14 and lower tray mount 26 (described below) to further increase the bite spacing between the upper and lower teeth—typically by approximately 5 mm—and additional spacers or ones of varied thicknesses can be used to properly set the desired bite spacing.

An upper tray mount 18 of the intra-oral device 10 is configured to accept the upper arch tray 12 (and optional spacer 16) within a crescent groove 20 defined along a topside thereof and includes a distally-extending frame 22 that terminates in a top-half 24 of a conical threaded compression fitting—best seen in FIG. 3. Similarly, a lower tray mount 26 is configured to accept the lower arch tray 14 therein and includes a distally extending frame 28 that terminates in a bottom-half 30 of the conical threaded compression fitting. The top-half 24 and bottom-half 30 each include substantially hemispherical depressions—e.g., depression 32 in the lower-half 30 of the conical threaded compression fitting—that when fitted together form a substantially spherical depression sized and configured to receive a ball 34 therebetween to form a ball-joint of the device. Top- and bottom-half 24, 30 are preferably fitted together using a pin-and-post arrangement 33 as shown in the drawings.

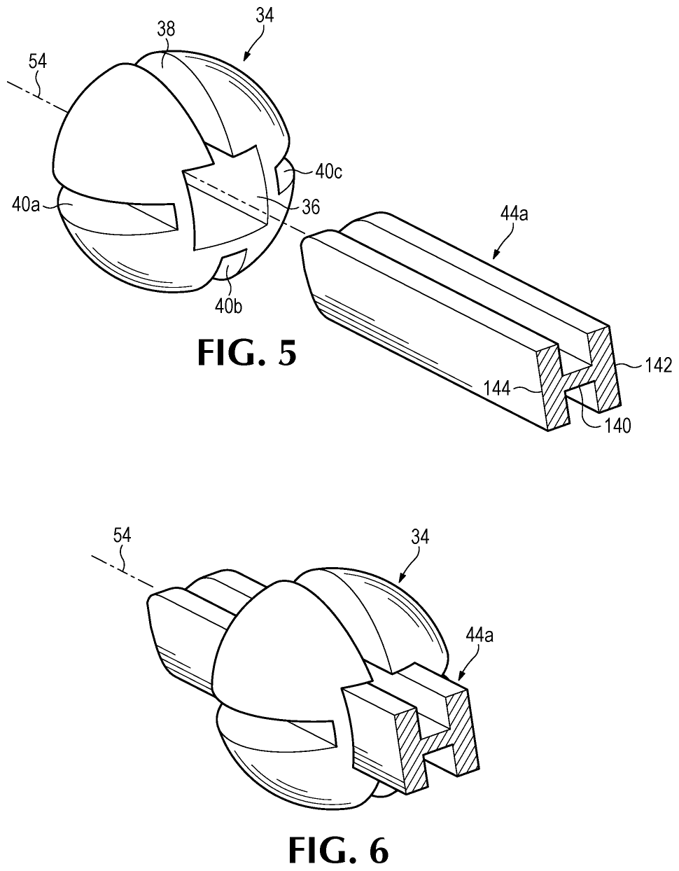

Ball 34 includes an aperture 36 passing axially through the ball between proximal (facing toward the patient) and distal (facing away from the patient) ends. Ball 34 further includes a main slot 38 running parallel with the aperture 36 and passing from an exterior surface of the ball to the interior of the aperture 36. Finally, ball 34 includes one or more flexures—shown in FIG. 5 as grooves 40a, 40b, and 40c—defined on the surface of the ball 34 and running in parallel to main slot 38.

Each arch tray can alternately include “teeth” formed on both sides, e.g. complementary pin and aperture structures on opposed sides of the arch trays 12, 14. When both arch trays 12, 14 are connected to form the device, being the mirror image, they snap into each other and prevent the ball 34 from torquing the front part of the arch tray sideways (this is the portion where the EVA dental bite occurs). Without this feature, the two sides of the trays may be offset slightly in opposing directions from this torquing. These structures can be formed proud of each of the inner facing surfaces of the trays 12, 14 so that when the arch trays are fitted together there is a space between then sufficient to allow the elongated rod 44a to extend between them.

The intra-oral device includes a protective element 42a formed with an elongate rod 44a that is received through the ball aperture 36 and is rotatable within the ball joint formed by the ball 34 within the depression 32. The elongate rod 44a terminates proximally in a paddle 46 that forms the head of the protective element 42a and includes a substantially planar portion 47 aligned with a long axis of the rod. Paddle 46 is configured to be extensible into the mouth of the patient as the rod 44a slides longitudinally within the aperture 36 of ball 34 and is rotatable via the ball joint through a plurality of positions against a tongue of the patient. Paddle 46 may optionally be fitted with a paddle extension 48 that snaps onto a terminal end of the paddle 46 to increase the size of the terminal end of the protective element as needed. Alternately, paddle 46 is comprised of a universal left/right paddle that includes a concave depression across one face 47 for a depressing paddle shape. The protective element 42a can then be oriented so that the concave depression is on the left or right side of the paddle 46 by removing from within the intra-oral device 10 and rotated 180 degrees so that the paddle depression faces the other side once reinserted through ball aperture 36 and extended into the mouth of the patient.

An alternate embodiment of the protective element is shown by protective element 42b, which is formed with an elongate rod 44b and terminates in a push-pull paddle 50 having an angled surface 52 defining an orthogonal plane to a long axis 54 of the rod 44b. Whereas paddle 46 of protective element embodiment 42a is best configured to push the patient's tongue to one side or the other of the mouth when the intra-oral device 10 is installed in the front of the mouth, the push-pull paddle 50 of protective element embodiment 42b is used best when the intra-oral device is installed in the side of a patient's mouth so that the tongue can be dragged toward the device or pushed away from the device as needed.

Intra-oral device 10 includes a compression fitting disposed between the upper and lower dental trays 12, 14. In a first embodiment, shown in FIGS. 1 and 3, compression fitting includes a taper nut 56 formed with interior conical threads 58 that are configured to engage with the exterior threads of the upper and lower conical threading pieces 24, 30. That is, when the upper and lower arch trays 12, 14 are fitted together so that the ball 34 is captured within the depressions formed within the upper and lower frames 22, 28, the dimension of the exteriorly threaded portion can be received within the taper nut 56.

An exterior plate 60 is coupled to a rear of the taper nut 58 and includes spikes 62, 64 extending distally that are configured to allow a user to rotate the plate 60 and coupled taper nut 58 to thereby tighten it against the exterior threaded portions 24, 30 of the upper and lower arch frames 22, 28. Alternate means for facilitating the tightening of the taper nut 58 include structures where the tapered nut includes four wings that protrude to allow for better ergonomic gripping to tighten and secure the paddle 42a in the desired position. They are also intended to secure a thermoformed face mask that is fitted to the patient after they have the device fitted and in the mouth. The upper portion of the wing is undercut, meaning it tapers down as you get close to the ball, which allows the thermoformed mask (separate device and step) to be pinched around the wings. The tapered element prevents the pinched mask from separating from the stent and will not allow the mask to move away from the face. The tapered nut can also have a “stop” so that it cannot be unthreaded entirely. It could be forced off but when loosened fully it will hit a stop under normal use conditions.

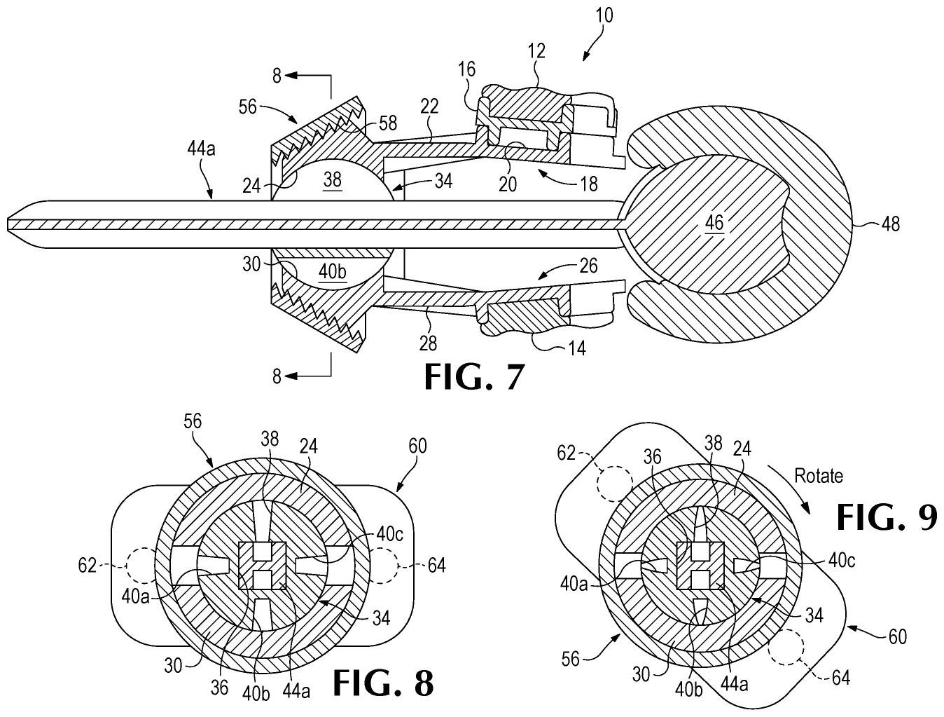

As will be appreciated by further description below, particularly with reference to FIGS. 7-9, turning the exterior plate 60 allows one to draw the exterior threaded portions 24, 30 into the narrowing interior of the taper nut 56, which thereby pinches the threaded portions 24, 30 together and deforms the ball 34 so that the sidewalls of the interior aperture 36 of ball 34 compresses against the rod (either embodiment 44a or 44b) of protective elements 42a, 42b and thereby fixes them in place. The flexure structure of the ball 34 allows the ball to be resiliently biased to a resting dimension but can be pinched by the compression fittings to fix the tongue-manipulating protective element as described below. In order to provide additional slippage protection when the paddle is being “crushed” when locked, ball 34 can be coated with a very thin urethane coating that adds a friction element between the ribs and the ball exterior. There may also be a compliance (squishiness) of the urethane coating when “crushed” which might help increase grip and retain the desired position and eliminate or reduce slippage as well.

Device 10 is inserted into the mouth of the patient so that a proximal side—characterized by the right side of FIG. 1—is inserted first, with the distal side to the left of the arches 12, 14 remaining outside of the patient's mouth. Arches 12 and 14 are preferably configured to accept only a partial arc of the upper and lower teeth, respectively, so that the intra-oral device 10 can be moved laterally along the arc of the patient bite. The paddles 46 or 50 may then be extended further into the interior of the patient's mouth and the lateral position manipulated by movement of the rods 44a or 44b within the ball joint formed by ball 34 and depression 32.

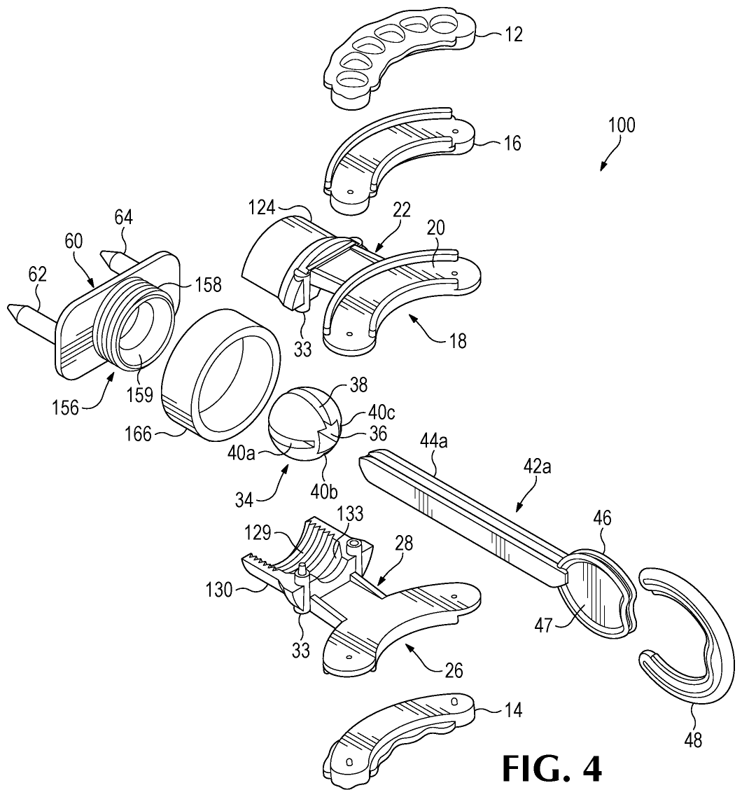

FIGS. 2 and 4 illustrate a second embodiment of an intra-oral device 100 configured according to the present invention with a tension ring forming a compressible fitting (described further below), where like part numbers denote like parts to the first embodiment 10. Intra-oral device 100 includes an upper arch tray 12 upon which a patient bites down with at least a portion of their maxillary teeth or maxillary edentulous arch as shown in FIG. 14. Device 10 further includes an opposed lower arch tray 14 configured to receive at least a portion of the patient's mandibular teeth or mandibular edentulous arch, again as shown in FIG. 14. An upper spacer 16 is optionally fitted to the upper arch tray 12 (e.g., between the upper arch tray 12 and upper tray mount 18) to increase the bite spacing between the upper and lower teeth. An elastomeric impression material such as EVA can be fitted within the upper and lower arch trays, or are in fact the trays 12, 14 themselves, to form fit around and engage the teeth of the patient for a more comfortable and more secure fit. Preferably, the EVA material is soaked in hot water to soften the EVA and to allow custom impression of patient's teeth. Once cooled, this allows the device to be indexed precisely in the same position during each treatment session over the course of 6-7 weeks of use.

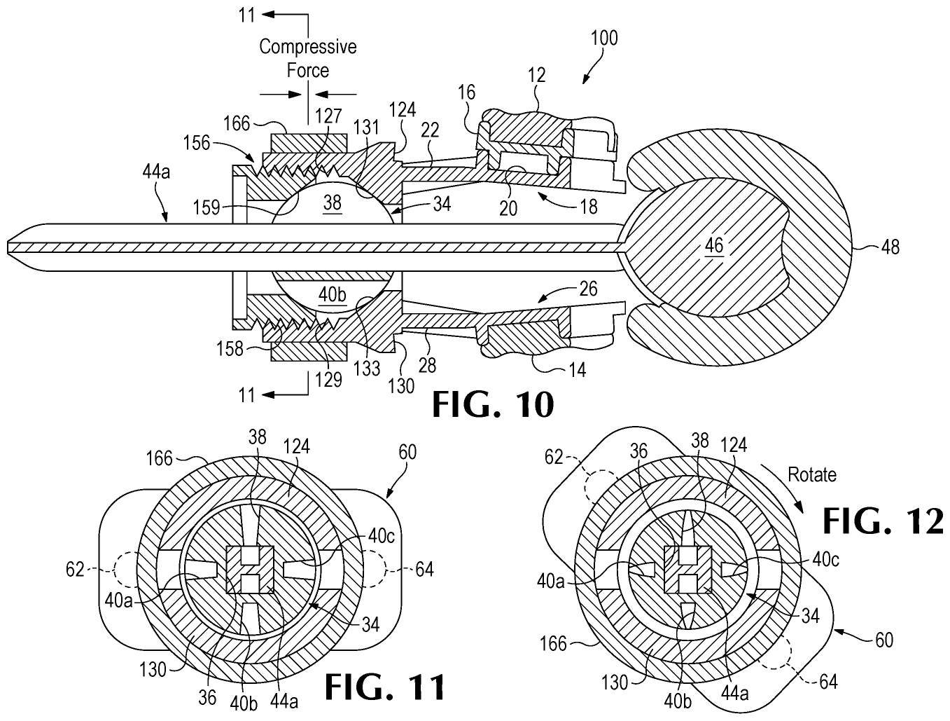

An upper tray mount 18 of the intra-oral device 100 is configured to accept the upper arch tray 12 (and optional spacer 16) within a crescent groove 20 defined along a topside thereof and includes a distally-extending frame 22 that terminates in a top-half 124 of a threaded compression fitting—best seen in FIG. 4. Similarly, a lower tray mount 26 is configured to accept the lower arch tray 14 therein and includes a distally extending frame 28 that terminates in a bottom-half 130 of the threaded compression fitting. As seen best in FIG. 4, the top-half 124 and bottom-half 130 each include interior threads 127, 129 (FIG. 10) bound on a proximal side by angled compressive surfaces 131, 133 that when fitted together form a substantially hemispherical depression sized and configured to receive a ball 34 therebetween to form a ball-joint of the device. Top- and bottom-half 124, 130 are preferably fitted together using a pin-and-post arrangement 33 as shown in the drawings.

Ball 34 includes an aperture 36 passing axially through the ball between proximal (facing toward the patient) and distal (facing away from the patient) ends. Ball 34 further includes a main slot 38 running parallel with the aperture 36 and passing from an exterior surface of the ball to the interior of the aperture 36. Finally, ball 34 includes one or more flexures—shown in FIG. 5 as grooves 40a, 40b, and 40c—defined on the surface of the ball 34 and running in parallel to main slot 38.

The intra-oral device 100 includes a protective element 42a formed with an elongate rod 44a that is received through the ball aperture 36 and is rotatable within the ball joint formed by the ball 34 within the ball joint cavity. The elongate rod 44a terminates proximally in a paddle 46 that forms the head of the protective element 42a and includes a substantially planar portion 47 aligned with a long axis of the rod. Paddle 46 is configured to be extensible into the mouth of the patient as the rod 44a slides longitudinally within the aperture 36 of ball 34 and is rotatable via the ball joint through a plurality of positions against a tongue of the patient to move the tongue to the right or left side of the mouth, and away from the irradiated area, as described further below. Paddle 46 may be fitted with a paddle extension 48 that snaps onto a terminal end of the paddle 46 to increase the size of the terminal end of the protective element as needed.

Intra-oral device 100 includes a compression fitting disposed between the upper and lower dental trays 12, 14. In the second embodiment, shown in FIGS. 2 and 4, the compression fitting includes an annular portion 156 extending proximally from a proximal surface of exterior plate 60. Annular portion 156 includes exterior threads 158 thereon that are configured to engage with the interior threads formed on the top- and bottom-half 124, 130 of the compression fitting, and an annular distal angled compressive surface 159 formed interiorly within the annual portion 156 that is configured to work cooperatively with angled proximal surfaces 131, 133 to form a substantially spherical cavity into which ball 34 sits. Ball 34 can then rotate within this cavity to form a ball joint capable of moving the rod 44a of the protective element through any desired angle within the mouth of the patient.

An exterior plate 60 is coupled to a rear of the annular portion 156 and includes spikes 62, 64 extending distally that are configured to allow a user to rotate the plate 60 and coupled annular portion 156 to thereby tighten it against the interior threads 127, 129 of top-half 124 and bottom-half 130 of the compressive fitting. As will be appreciated by further description below, particularly with reference to FIGS. 10-12, turning the exterior plate 60 screws in the threaded portions and allows one to draw the angled proximal surfaces 131, 133 closer to the annular distal angled compressive surface 159, which thereby pinches these portions together and deforms the ball 34 so that the sidewalls of the interior aperture 36 of ball 34 compresses against the rod 44a of protective elements 42a and thereby fixes it in place. A tension ring 166 is fitted around the exterior of the top-half 124 and bottom-half 130 of the compressive fitting to keep these structures from expanding as the compression fitting is tightened. The flexure structure of the ball 34 allows the ball to be resiliently biased to a resting dimension but can be pinched by the compression fittings to fix the tongue-manipulating protective element in place as described below.

Alternate means for facilitating the tightening of the annular portion 156 within the compression fittings 124, 130 include structures where the plate 60 includes four wings that protrude to allow for better ergonomic gripping to tighten and secure the paddle 42a in the desired position. They are also intended to secure a thermoformed face mask that is fitted to the patient after they have the device fitted and in the mouth. The upper portion of the wing is undercut, meaning it tapers down as you get close to the ball, which allows the thermoformed mask (separate device and step) to be pinched around the wings. The tapered element prevents the pinched mask from separating from the stent and will not allow the mask to move away from the face.

Device 100 is inserted into the mouth of the patient so that a proximal side—characterized by the right side of FIG. 2—is inserted first, with the distal side to the left of the arches 12, 14 remaining outside of the patient's mouth. Arches 12 and 14 are preferably configured to accept only a partial arc of the upper and lower teeth, respectively, so that the intra-oral device 10 can be moved laterally along the arc of the patient bite. The paddle heads, either embodiments 46 or 50, may then be extended further into the interior of the patient's mouth and the lateral position manipulated by movement of the rods 44a or 44b within the ball joint formed by ball 34 and spherical cavity.

FIGS. 5 and 6 show magnified views of the ball 34 engaged with the rod 44a of the protective element 42a (rod 44b of protective element 42b would work similarly). Rod 44a is preferably formed with a reinforced I-beam shape cross-section as shown in the figures, including a central webbing 140 coupling between parallel flanges 142, 144. The I-beam cross section is easily slidingly received within the square cross-section of ball aperture 36 (FIG. 6), wherein pinching of the ball using the compressive fittings noted above and further described below cause the aperture size to shrink and thereby pinch the rod 44a to make it more resistant to sliding movement within the aperture 36.

FIGS. 7-9 illustrate cross-sections of the first embodiment 10 of the intra-oral device and show movement of the compressive fittings between a first/loosened position and second/tightened position to fix the ball 34 and inserted rod 44a in place. FIGS. 7 and 8 show side elevation and end cross-sections, respectively, of intra-oral device 10 with the threads of upper and lower halves 24, 30 in a first-threaded position with respect to taper nut 56. In this first position, the threads are relatively untightened so that the ball 34 is maintained in its normal, unbiased position and so that the slots—including main slot 38 and flexures 40a, 40b, and 40c—are in their undeformed/open position. As plate 60 is rotated as shown in FIG. 9, however, the threads of the upper and lower halves 24, 30 are drawn further into the taper nut 56 and pressure is placed on ball 34 to close the ball's dimensions around slot 38 and flexures 40a, 40b, and 40c. This causes the inner surfaces of the ball aperture 36 to pinch the protective element rod 44a to make it more difficult to slide the rod in and out of the aperture 36. The pressure placed on the ball 34 also prevents the ball from freely rotating within the substantially spherical depression of the ball joint to thereby not only fix the rotational angle of the protective element in place, but also its extension distance into the mouth of the patient.

FIGS. 10-12 illustrate cross-sections of the second embodiment 100 of the intra-oral device showing movement of the compressive fittings in order to fix the ball 34 and inserted rod 44a in place. FIGS. 10 and 11 show side elevation and end cross-sections, respectively, of intra-oral device 100 with the threads of upper and lower halves 124, 130 in a first-threaded position with respect to the threaded distal annual portion 56. In this first position, the threads are relatively untightened so that the ball 34 is maintained in its normal, unbiased position and so that the slots—including main slot 38 and flexures 40a, 40b, and 40c—are in their undeformed/open position. As plate 60 is rotated as shown in FIG. 12, however, the front and back of the intra-oral device 100 are drawn closer together so that the proximal angled surfaces 131, 133 and distal angled surface 159 apply increasing pinching force to the ball 34 between them to close the ball's dimensions around slot 38 and flexures 40a, 40b, and 40c. This causes the inner surfaces of the ball aperture 36 to pinch the protective element rod 44a to make it more difficult to slide the rod in and out of the aperture 36. The pressure placed on the ball 34 also prevents the ball from freely rotating within the substantially spherical depression of the ball joint to thereby not only fix the rotational angle of the protective element in place, but also its extension distance into the mouth of the patient. In an alternate embodiment, ball 34 could include “crushed ribs” that are configured to be compressed (complied) when the taper nut is tightened over it. These ribs have an additional advantage in that they help prevent the ball from slipping when the tongue rests and applies force to the paddle head. The universal ball 34 can alternately include ribs that are ˜90 degrees from the arch tray threads. This will allow the ball to “crush” into the tray threads and create a “lock” to prevent movement of the positioned paddle more effectively.

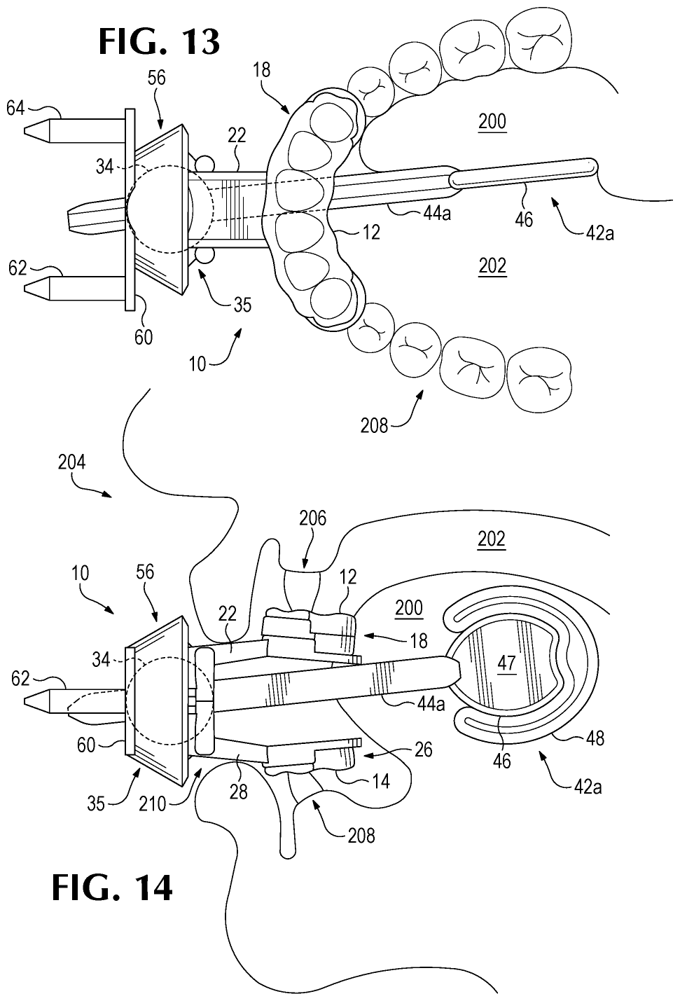

FIGS. 13 and 14 illustrate a preferred method for using the intra-oral device. Whereas these images show use of the first embodiment 10, it is understood that such methods could be applied to any such devices configured according to teachings of the invention including the second embodiment 100. Intra-oral device 10 allows one to reposition the tongue 200 within the oral cavity 202 of a patient 204 with or without maxillary 206 and/or mandibular teeth 208. The preferred method comprises installing the intra-oral device 10 in the mouth 210 of the patient 204 so that oral arches of the patient engage with an upper dental arch member 18 and a lower dental arch member 26 of the intra-oral device 10.

In the drawings shown, the device 10 is engaged only with the patient's incisors at the front of the mouth. However, the device lower arch tray 14 may also be moved along right and left sides of the mandibular arch so as to engage canines, premolars, and molars so that the protective element approaches the tongue from the side. The upper arch tray 12 may be similarly adjusted.

The protective element 42a is then moved within a ball joint 35 coupled to a distal portion of the intra-oral device so that an extensible portion of the protective element bears proximally against the tongue 200 of the patient 204 and moves the tongue from a central axis of the oral cavity 202. The step of moving the protective element 42a includes capturing a ball 34 of the ball joint 35 within a compressible fitting between the oral arches, wherein the ball includes an aperture 36 therethrough passing from a proximal to a distal portion. The rod 44a of the protective device 42a is then extended through the aperture 36 of the ball 34 and rotated through the ball joint until the extensible portion is in a desired position adjacent a tongue of the patient. FIG. 13 shows the protective device 42a bearing against a left side of the tongue 200 and moving the tongue to the far-right side of the oral cavity 202. The compression fitting is then compressed via methods described above so that a dimension of the ball 34 is pinched within the compression fitting to thereby prevent the ball from rotating within the ball joint and to thereby fix the protective device at the rightward angle shown. Operation of the compression fitting further pinches the aperture closed about the rod extended through the aperture to thereby fix the extensible portion in position within the mouth of the patient. By fixing the distance and angle of the protective device, the patient's tongue is thus secured at a desired location away from where other oral tissues might be irradiated for medical imaging.

Each product kit will come with two spacers. The arch tray, spacer and EVA pieces have been re-designed to all universal snap on/off so that should a clinic need to open the mouth further for treatment, they can a) remove the EVA piece and add one or two spacers to the tray followed by the EVA insert on top (see assembly image showing two spacers added). They can also choose to add one additional spacer to each top and bottom arch tray as needed. There is flexibility built in here. The receptacles in arch tray, spacer and EVA insert are designed for secure attachment but not too secure that it could not be removed (will require a flathead screwdriver for example to pry open).

In use, the stent device is removed from its packaging and the stent arch tray heated in order to soften the insert material so that a bite impression can be made. In one method, water is heated to >80 degrees Celsius and the arch trays soaked for 2 minutes. Alternately, the stent arch trays are heated in an oven at >80 degrees Celsius for six minutes. The heated stents are then removed from the heat and rested for ten seconds before placing the heated arch tray into a patient's mount. The patient would then bite down onto the stent insert material for fifteen seconds to form teeth impressions before removing. The arch trays are then rinsed in cold water to set the impressions.

In the next step, a desired paddle is selected—e.g. the depressing paddle or sideways deviating paddle—and the paddle inserted into the central anterior groove of the arch tray, with the smooth, concave side of the paddle located on the tongue-facing side. The paddle is then aligned by sliding it back and forth within the anterior/posterior plane and the paddle angled up, down or sideways as desired to secure the tongue in the desired position. The paddle is then secured in place, e.g., by moderately tightening the anterior wing nut clockwise before placing the stent in the patient's mouth for evaluation. If necessary, the paddle is adjusted to achieve the desired paddle position to ensure patient comfort and tolerance. The paddle is then “locked” into position by further tightening the nut or ring clockwise so that the paddle is positioned without risk of loosening or slipping. As one tightens the locking device, one might hear a clocking sound, which indicates that the paddle is sufficiently tightened. The tongue is then deviated out of way so that imaging can be done of the mouth without exposing the tongue to unnecessary radiation.

In the foregoing description, numerous details have been set forth to provide a thorough understanding of the disclosed exemplary embodiments for an intra-oral device for positioning certain oral tissue during radiation treatment. The purpose of the intra-oral devices described here is to provide a wide range of flexibility to give the end user of the device as much latitude to customize and idealize its application for the maximum benefit of the patient. However, certain of the described details may not be required to provide useful embodiments, or to practice selected or other disclosed embodiments. Further, the description may include, for descriptive purposes, various relative terms such as surface, at, adjacent, proximity, near, on, onto, and the like. Such usage should not be construed as limiting. Terms that are relative only to a point of reference are not meant to be interpreted as absolute limitations but are instead included in the foregoing description to facilitate understanding of the various aspects of the disclosed embodiments. Various components are described which may be employed alternatively yet be included in a kit or product package to enable an end user to select the optimal components for use in a particular situation. Accordingly, procedures utilizing the intra-oral device described herein, and the method(s) described herein may be utilized as multiple discrete operations, in a manner that is most helpful in a particular circumstance. However, the order of description should not be construed as to imply that such alternatives are necessarily order dependent, or that use of various components is necessarily in the alternative. Also, the reader will note that the phrase “in one embodiment” has been used repeatedly. This phrase generally does not refer to the same embodiment; however, it may. Finally, the terms “comprising”, “having” and “including” should be considered synonymous, unless the context dictates otherwise.

Various aspects and embodiments described and claimed herein may be modified from those shown without materially departing from the novel teachings and advantages provided by this invention and may be embodied in other specific forms without departing from the spirit or essential characteristics thereof. Embodiments presented herein are to be considered in all respects as illustrative and not restrictive or limiting. This disclosure is intended to cover methods and apparatus described herein, and not only structural equivalents thereof, but also equivalent structures. Modifications and variations are possible considering the above teachings. Therefore, the protection afforded to this invention should be limited only by the claims set forth herein, and the legal equivalents thereof.

Having described and illustrated the principles of the invention in a preferred embodiment thereof, it should be apparent that the invention can be modified in arrangement and detail without departing from such principles. We claim all modifications and variation coming within the spirit and scope of the following claims.

What is claimed is:

1. An intra-oral device for positioning oral tissues in a patient, said device comprising:
an upper dental arch tray configured for engagement with the maxillary teeth or maxillary edentulous arch of a patient;
a lower dental arch tray configured for engagement with the mandibular teeth or mandibular edentulous arch of the patient;
a compression fitting disposed between the upper and lower dental arch trays;
a ball disposed and forming a rotatable ball joint within the compression fitting, said ball having an aperture therethrough; and
a protective element including a head and elongate rod, wherein the rod is received through the aperture of the ball and the head is configured to be extensible into the mouth of the patient and rotatable via the ball joint through a plurality of positions against a tongue of the patient and lockable in place via the compression fitting about the ball.

2. The intra-oral device of claim 1, wherein the ball is a compressible ball and includes slots form along an exterior of the ball.

3. The intra-oral device of claim 2, wherein the ball includes a urethane coating on an exterior surface thereof operative to increase a friction of the ball exterior surface within the compression fitting.

4. The intra-oral device of claim 2, wherein the ball includes a urethane coating on an exterior surface thereof operative to provide compliance (squishiness) of the ball exterior surface within the compression fitting.

5. The intra-oral device of claim 2, wherein the ball includes a main slot passing from an exterior of the ball to the aperture.

6. The intra-oral device of claim 1, further including a spacer disposed within at least one of the upper and lower dental arch trays.

7. The intra-oral device of claim 1, wherein the head of the protective element is a paddle having a substantially planar portion aligned with a long axis of the rod.

8. The intra-oral device of claim 6, further including a paddle extension coupled to an end of the paddle to increase a dimension of the paddle.

9. The intra-oral device of claim 1, wherein the head of the protective element is a push-pull paddle having an angled surface defining an orthogonal plane to a long axis of the rod.

10. The intra-oral device of claim 1, wherein the rod includes an I-beam cross-section.

11. The intra-oral device of claim 1, wherein the compression fitting includes a taper nut.

12. The intra-oral device of claim 11, further including:
opposed exterior threaded portions on each of the upper and lower arch trays;
hemispherical depressions interior of the exterior threaded portions such that assembly of the upper and lower arch trays result in a substantially spherical depression configured to capture the ball within,
wherein the taper nut is configured to engage with the exterior threaded portions of the upper and lower arch trays and compress the trays together to thereby compress the substantially spherical depression against the ball and lock the rotation movement of the ball in place and rod within the ball aperture.

13. The intra-oral device of claim 12, further including an exterior plate coupled to a rear of the taper nut and including spikes extending rearwardly and configured to allow a user to rotate the taper nut and tighten it against the exterior threaded portions of the upper and lower arch trays.

14. The intra-oral device of claim 12, further including a plurality of wings extending from an exterior surface of the taper nut configured to allow a user to rotate the taper nut and tighten it against the exterior threaded portions of the upper and lower arch trays.

15. The intra-oral device of claim 12, wherein the ball includes ribs configured approximately 90 degrees from the exterior threaded portions on each of the upper and lower arch trays.

16. The intra-oral device of claim 11, wherein the taper nut includes a stop configured to prevent the tapered nut from being unthreaded entirely from within the compression fitting.

17. The intra-oral device of claim 1, wherein the compression fitting includes a tension ring.

18. The intra-oral device of claim 17, further including:
opposed interior threaded portions on each of the upper and lower arch trays;
first compression surfaces proximal the dental arches relative to the interior threaded portions;
an exterior plate having exterior threaded surfaces thereon configured to threadably engage with the interior threaded portions of the upper and lower arch trays, and further including second compression surfaces proximal the first compression surfaces relative to the exterior threaded surfaces, wherein the first and second pressions surfaces form a substantially spherical depression therebetween,
wherein the tension ring is configured to engage around an exterior of the opposed interior threaded portions and prevent them from expanding as the exterior threaded surfaces are threaded into the interior threaded portions to thereby longitudinally contract the substantially spherical depression about the received ball and thereby lock the rotation movement of the ball in place and rod within the ball aperture.

19. The intra-oral device of claim 1, further including teeth formed on opposing sides of the arch trays so that the arch trays can be directly coupled together and thereby mitigate torquing of the arch trays relative to one another.

20. A method for repositioning the tongue within the oral cavity of a patient with or without maxillary and/or mandibular teeth, said method comprising:
installing an intra-oral device in the mouth of the patient so that oral arches of the patient engage with an upper dental arch member and a lower dental arch member of the intra-oral device;
moving a protective element within a ball joint coupled to a distal portion of the intra-oral device so that an extensible portion of the protective element bears proximally against the tongue of the patient and moves the tongue from a central axis of the oral cavity, the step of moving including:
capturing a ball of the ball joint within a compressible fitting between the oral arches, wherein the ball includes an aperture therethrough passing from a proximal to a distal portion;
extending a rod of the protective device through the aperture of the ball and rotating the protective element through the ball joint until the extensible portion is in a desired position adjacent a tongue of the patient;
compressing the fitting about the ball so that a dimension of the ball is pinched within the compression fitting to thereby prevent the ball from rotating within the ball joint, and further pinching the aperture closed about the rod extended through the aperture to thereby fix the extensible portion in position within the mouth of the patient so as to secure the patient's tongue at a desired location while providing radiation treatment to selected tissues.
